(19) United States
(12) Patent Application Publication
(10) Pub. No.: US 2024/0148802 A1
(43) Pub. Date: May 9, 2024

(54) MITIGATION OF PATHOGENS IN PORCINE SPECIES

(71) Applicant: Purina Animal Nutrition LLC, Arden Hills, MN (US)

(72) Inventors: Huyen T. Tran, Lincoln, NE (US); Aileen Joy Mercado, Woodbury, MN (US); Brenda de Rodas, O'Fallon, MO (US); Stacie Crowder, Arden Hills, MN (US)

(21) Appl. No.: 18/504,131

(22) Filed: Nov. 7, 2023

Related U.S. Application Data
(60) Provisional application No. 63/383,028, filed on Nov. 9, 2022.

(51) Int. Cl. A61K 35/742; A23K 10/18; A23K 20/111; A23K 20/121; A23K 20/158; A23K 50/30; A61K 9/00; A61K 31/192; A61K 31/20; A61K 31/593; A61K 47/18; A61K 47/46; A61P 31/04

(52) U.S. Cl. CPC A61K 35/742; A23K 10/18; A23K 20/111; A23K 20/121; A23K 20/158; A23K 50/30; A61K 9/0056; A61K 31/192; A61K 31/20; A61K 31/593; A61K 47/183; A61K 47/186; A61K 47/46; A61P 31/04; A61K 2035/115

(57) ABSTRACT

The present disclosure describes products formulated to mitigate viral and bacterial infections in swine, along with associated methods of providing such products to the animals. Feeding methods involve providing animals with a feed product that includes a medium chain fatty acid composition, a direct-fed microbial composition, along with one or more flavonoids and/or organic acids. The medium chain fatty acid composition includes one or more of caproic acid, caprylic acid, capric acid and lauric acid. The direct-fed microbial composition includes one or more strains of Bacillus. The animals can include swine at risk of developing, or already afflicted with, a respiratory condition such as porcine reproductive respiratory syndrome or pneumonia.

### CROSS-REFERENCE TO RELATED APPLICATIONS

This application claims priority to U.S. Provisional Patent Application No. 63/383,028, filed Nov. 9, 2022, entitled “MITIGATION OF PATHOGENS IN PORCINE SPECIES,” which is incorporated by reference herein, in the entirety and for all purposes.

### TECHNICAL FIELD

The present disclosure relates to compositions and methods for mitigating pathogens in porcine species, including bacterial pathogens and viral pathogens associated with porcine reproductive respiratory syndrome (PRRS). Implementations provide feed products containing active components in an amount effective to mitigate the impacts of viral and bacterial pathogens associated with PRRS upon ingestion.

### BACKGROUND

Porcine reproductive respiratory syndrome, caused by the porcine reproductive respiratory syndrome virus (PRRSV), consistently leads to significant losses in the U.S. swine industry. In the U.S. alone, the annual economic losses due to PRRS are estimated to be approximately $664M in direct cost and $447M in indirect cost. Over 60% of PPRS infection is concurrent with other pulmonary viral and bacterial pathogens, which lead to respiratory disease complex. The combined effect of PRRS and one or more additional respiratory conditions caused at least in part by bacterial pathogens, e.g., Streptococcus suis and pneumonia, may be especially harmful. Currently, vaccination against PRRSV provides limited protection to prevent disease outbreak and mortality, and little to no benefit for combating conditions caused by bacterial pathogens often responsible for additional respiratory conditions. Many pigs grown for commercial purposes become infected with the virus and various bacterial pathogens every year, and current methods of treatment are ineffective. PRRS virus is a highly infectious airborne disease and can be transmitted through contacting excretion such as feces, semen, urine, mammary and nasal secretions. In addition, the conditions in which pigs are often raised, e.g., indoor facilities or outdoors, further hinder efforts to slow transmission of the virus and associated bacteria, leaving swine producers with few options for reducing the likelihood of their animals becoming infected. Accordingly, new methods are needed to prevent or at least reduce the impact of PRRS and various bacterial conditions without compromising animal performance.

### SUMMARY

In accordance with embodiments of the present disclosure, a method of mitigating pathogenic infection in an animal involves admixing a mitigant composition with a base feed to form a mitigant feed mixture. The mitigant composition may include a medium chain fatty acid composition and a direct-fed microbial composition. The direct-fed microbial composition may include two or more strains of Bacillus selected from strains of Bacillus pumilus, Bacillus subtilis, and Bacillus amyloliquefaciens. The medium chain fatty acid composition may include one or more of caproic acid, caprylic acid, capric acid, or lauric acid. The method may further involve providing the mitigant feed mixture to the animal in an amount effective to mitigate the pathogenic infection.

In some examples, the mitigant feed mixture is provided on an ad libitum basis. In some examples, the mitigant composition further includes one or more flavonoids. In some examples, the one or more flavonoids include a citrus flavonoid. In some examples, the animal is a nursery pig. In some examples, the mitigant composition further includes one or more organic acids, which may include benzoic acid.

In some examples, the pathogenic infection is a porcine reproductive respiratory syndrome virus infection. In some examples, the pathogenic infection is a Streptococcus suis infection. In some examples, the pathogenic infection includes both a PRRSV infection and a S. suis infection.

In some examples, the base feed includes a complete swine feed, blood meal, porcine meat and bone meal, spray-dried animal plasma, feather meal, avian blood meal, poultry by-product meal, vitamin D, lysine hydrochloride, choline chloride, and/or soybean meal. In some examples, the two or more strains of Bacillus are included in approximately equal amounts. In some examples, the direct-fed microbial composition constitutes about 0.1 wt % to about 5.0 wt %, or to about 10.0 wt % of the mitigant composition. In some examples, the medium chain fatty acid composition constitutes about 20 wt % to about 60 wt % of the mitigant composition. In some examples, the animal ingests about 0.1 g to about 5.0 g of the mitigant composition per day. In some examples, the inclusion rate of the mitigant composition in the base feed is about 1 lb./ton to about 20 lbs./ton. In some examples, providing the mitigant feed mixture to the animal in an amount effective to mitigate the pathogenic infection involves providing the mitigant feed mixture to the animal in an amount effective to reduce one or more symptoms of PRRS in the form of respiratory distress, weight loss, decreased feed intake, or increased feed-to-gain ratio. In some examples, providing the mitigant feed mixture to the animal in an amount effective to mitigate the pathogenic infection involves providing the mitigant feed mixture to the animal in an amount effective to reduce PRRS viral load in serum and lung tissue of the animal.

In accordance with embodiments of the present disclosure, a feed product formulated for swine may include a complete base feed comprising one or more of: ground corn, sugar, dehulled soymeal, soy isolate, hydrolyzed soy protein, dried distillers grains with solubles, poultry byproduct meal, vitamins, or minerals. The feed product may also include at least one direct-fed microbial comprising two or more strains of Bacillus selected from strains of Bacillus pumilus, Bacillus subtilis, and/or Bacillus amyloliquefaciens, as well as at least one medium chain fatty acid comprising one or more of caprylic acid, capric acid, or lauric acid.

In some examples, the feed product may further include one or more flavonoids, which may include a citrus flavonoid. In some examples, the feed product further includes one or more organic acids, which may include benzoic acid. In some examples, the feed product also includes one or more anti-caking agents, flavorants, desiccants, emulsifiers, preservatives, stabilizers, salts, or combinations thereof.

### BRIEF DESCRIPTION OF THE DRAWINGS

FIG. 1 is a flow diagram of a method of mitigating pathogens in accordance with principles of the present disclosure.

FIG. 2 is a table showing the relative percentages of nursery pigs challenged with PRRSV and S. suis exhibiting respiratory distress both with and without consuming a feed product disclosed herein.

FIG. 3 is a table showing the relative PRRS viral loads present in the serum and lungs of nursery pigs challenged with PRRSV and S. suis both with and without consuming a feed product disclosed herein.

FIG. 4 is a table showing the relative percentages of nursery pigs challenged with PRRSV and S. suis having at least one pneumonia lesion both with and without consuming a feed product disclosed herein.

FIG. 5 is a table showing the relative percentages of lungs with pneumonia in nursery pigs challenged with PRRSV and S. suis both with and without consuming a feed product disclosed herein.

FIG. 6 is a table showing the performance improvements exhibited by nursery pigs challenged with PRRSV and S. suis fed a feed product disclosed herein relative to co-infected pigs not fed the feed product.

FIG. 7 is a table showing the impact of consuming a disclosed feed product on Mycoplasma, Streptococcus, Staphylococcus, and Lactobacillus levels in nursery pigs challenged with PRRSV and S. suis.

### DETAILED DESCRIPTION

Implementations provide methods of mitigating viral and bacterial infections often associated with PRRS by feeding or otherwise orally administering animals afflicted with or susceptible to the syndrome a feed product containing one or more medium-chain fatty acids (MCFAs), direct-fed microbials (DFMs), flavonoids, and/or organic acids. The feed product can be a stand-alone product, a product formulated for mixing with base feed or liquid, and/or an integral component of a base feed or liquid. The animals offered the feed products disclosed herein can include various types of swine across a broad age range. The feed products can be administered before or after the animals exhibit symptoms indicative of a respiratory condition, such as coughing or labored breathing. By not relying on the use of harmful chemicals and antibiotics, the disclosed feed products and associated methods provide safe, natural alternatives to preexisting approaches of inhibiting respiratory stress conditions. Examples of the feed products may even be specifically non-medicated, meaning the compositions lack one or more additional antiviral agents, antibiotics, or medicinal compositions. Embodiments may also be more effective than traditional respiratory treatment approaches, evidenced by the significant improvements in animal health achieved upon implementation of the disclosed methods.

The disclosed feed products and feeding methods are effective to mitigate PRRS and other respiratory conditions, such as pneumonia, including symptoms associated therewith. As used herein, “mitigation” may encompass the prevention, control, reduction, or elimination of a viral and/or bacterial infection that causes or is associated with a respiratory condition, such as PRRS. Mitigation may also encompass treatment of PRRS and bacterial infections evidenced by a reduction in the severity, duration, and/or number of symptoms exhibited by an animal diagnosed with PRRS and other respiratory conditions. For example, mitigation may refer to a reduction of nasal pathogens during and after PRRSV infection, along with a reduction of the negative impacts of PRRS on animal performance, such that mitigated animals exhibit improved body weight, average daily gain, average daily feed intake, and feed-to-gain ratio relative to non-mitigated animals infected with PRRSV and one or more bacterial pathogens, such as Streptococcus suis. Mitigation may also refer to a reduction in the incidence and/or severity of pneumonia, respiratory distress, and viral loads in both serum and lung tissue of animals infected with PRRSV and one or more bacterial pathogens, e.g., S. suis. Mitigation may also refer to a maintenance of nasal and cecal bacteria beneficial to animals after infection. Mitigation may refer to a reduction in the measurable levels of one or more bacterial pathogens and/or a decrease in the growth rate of such pathogens present within an animal relative to an untreated control animal subjected to the same pathogens. Mitigation may also include a reduction or elimination of one or more associated symptoms, e.g., coughing, panting, or shallow breathing. Mitigation may encompass the prevention of PRRS, especially for animals at a heightened risk of contracting it, for example animals not diagnosed with or currently exhibiting respiratory symptoms, but exposed to PRRSV and bacteria commonly associated with the condition, e.g., S. suis, Mycoplasma spp., Actinobacillus spp., and Pasteurella spp. At-risk target animals may also include animals maintained in a setting where the incidence of respiratory conditions is moderate or high, and/or animals kept in a location where weather conditions are more favorable to the development of respiratory conditions. At-risk target animals may also include otherwise stressed animals and/or animals with compromised immunity. As a result of the mitigation achieved via implementation of the disclosed embodiments, mortality and morbidity rates may be reduced.

As used herein, an “effective amount” refers to an amount capable of providing bioavailable levels of the active components sufficient to mitigate PRRS and one or more additional conditions caused by viral and/or bacterial pathogens in swine. The active components included in a disclosed feed product may include one or more MCFAs, DFMs, flavonoids, and/or organic acids. The term “feed product” may refer to or be used interchangeably with “mitigant composition.” A feed product or mitigant composition may be defined as the composition of active components, which may also include one or more carrier components. The disclosed feed products may be formulated for inclusion with one or more base feeds, the resulting combination of which may be referred to as a mitigant feed mixture in some examples.

The terms “base feed,” “basal feed,” “total feed,” “starter feed,” and “complete feed” may be used interchangeably herein. Such terms encompass feeds into which the disclosed feed products may be admixed in certain embodiments. Examples of the aforementioned feeds may be provided concurrently with one or more additional feeds or milk replacers, or feeds that provide all or a majority of the targeted animals' nutrition, e.g., complete feeds.

The term “base liquid” encompasses a variety of liquids with which the disclosed feed products can be mixed. Non-limiting examples of base liquids can include drinking water, drenches, liquid milk replacers, or milk products comprised of natural and/or supplemental milk components.

As used herein, “pathogens” encompasses both viral and bacterial pathogens. Viral and bacterial infection may occur simultaneously or successively in any order, such that viral infection may precede bacterial infection, or vice versa. Viral infection may increase the risk of bacterial infection, or vice versa.

Animal species fed the feed products disclosed herein may vary, and may include a variety of animals including animals housed in group settings, such as in farms or research facilities, animals housed individually, and animals susceptible to respiratory infections such as animals fed during seasonal changes, or during fall, winter, or spring; and sick animals. Such animals may include swine raised for pork production, including but not limited to nursery pigs. Some embodiments may be specific to nursery pigs, piglets or otherwise young pigs. Young pigs may include those that are pre-weaned, weaning, or weaned, including recently weaned pigs, e.g., pigs weaned about 7, about 14, about 21, about 28, or about 35 or more days prior to the onset of feeding. Embodiments specific to nursery pigs may involve feeding a disclosed feed product to the nursery pigs beginning around 18-28 days of age, and continuing for at least about 1 week, about 2 weeks, about 3 weeks, about 4 weeks, about 5 weeks, or about 6 weeks thereafter, or any length of time therebetween. However, the feed products may be fed to pigs from 0 days to about 12 months of age, 0 days to about 6 months of age, 1 week to about 6 months of age, 1 week to about 7 months of age, 2 weeks to about 3 months of age, 2 weeks to about 7 months of age, or starting at about 0 day from birth, 1 day from birth, about 1 week from birth, about 2, 3, 4, 5, 6, 7, 8, 9, 10, 11 or 12 weeks from birth. Embodiments may also include or be specific to weaned pigs, such as wean-to-finish pigs, which may include pigs at an age between about weaning and slaughter, encompassing up to about 4 weeks post-weaning, about 5 weeks post-weaning, about 6 weeks post-weaning, about 7 weeks post-weaning, about 8 weeks post-weaning, about 9 weeks post-weaning, about 10 weeks post-weaning, or older, depending in part on the age of the pigs at the onset or end of the finishing phase. Solely for ease of illustration and to encompass all contemplated target animals, this disclosure refers simply to “animals” as the beneficiaries of the disclosed feed products and associated methods.

### Feed Products Formulated to Mitigate PRRS and Other Pathogenic Conditions

Embodiments of the disclosed feed products may include at least one MCFA, at least one DFM, at least one flavonoid, and/or at least one organic acid, which may be collectively referred to as the “active components” constituting all or a portion of a given feed product. The feed products can comprise stand-alone products, supplements, or “add-packs.” Additionally or alternatively, the feed products can be formulated for inclusion or admixing with a base feed or feed ingredients, drinking water, feed gel, and/or milk replacers before or after aqueous reconstitution. Accordingly, the feed products disclosed herein may be formed into or obtained as a supplement, additive, premix, pack, or integral component of a base feed, complete feed, liquid, and/or gel.

As a supplement or pack, the feed products may be provided to animals separately from or together with other feeds, feed ingredients, or liquids. As an additive, the feed products may be included within or admixed with a base feed, which may constitute a complete feed or a starter feed, such that the feed products each constitute a subcomponent of a broader feed composition. As a premix, the feed products may be combined with one or more additional vitamins, minerals, or additives, and the resulting composition may be formulated for admixing with a base feed or liquid. Feed products intended for admixing with a liquid or gel may be formulated differently than feed products intended for inclusion within a dry or substantially dry base feed. For example, such feed products may be water-soluble. Additives or premixes may be formulated for combining with a feed or liquid composition at or before the time of feeding. The feed products may provide little to no nutritional benefit beyond the inhibition of a respiratory condition, such that the base feed or liquid into which the feed products are added may provide the majority or sole source of nutrients to the target animals. Alternatively, embodiments of the disclosed feed products may provide one or more benefits beyond the inhibition of a respiratory condition, such as an improvement in the overall health or performance of an animal that ingests the feed products. The physical form of the feed products may vary and may depend on whether they are utilized as a supplement, pack, additive, or premix. Embodiments may include a dry feed product, e.g., a powder or granular composition. Delivering the mitigant compositions via a feed and/or liquid composition may be easier and more effective than other routes of administration, e.g., via medicinal boluses or intravenous injection.

Embodiments of the feed product may be customized to particular animals, such as piglets, recently weaned pigs, growing phase pigs, and/or finishing phase pigs. The pig or piglet feed product may include one or more MCFAs, DFMs, and flavonoids, but may exclude one or more organic acids in some examples. In embodiments, the same or similar feed product may be offered to piglets, recently weaned pigs, growing phase pigs, and/or finishing phase pigs. The MCFA(s) included in the disclosed feed products may include a single MCFA or a blend of two or more MCFAs including but not limited to: caproic acid (C6), heptanoic acid (C7), caprylic acid (C8), nonanoic acid (C9), capric acid (C10), undecanoic acid (C11) and/or lauric acid (C12). A single MCFA or a blend of two or more MCFAs may be included in the feed products and may be referred to as an “MCFA composition” regardless of the number of unique MCFAs included therein. For example, embodiments of the MCFA composition may include only caproic acid, only caprylic acid, only capric acid, or only lauric acid. Embodiments may specifically exclude caproic acid, for instance, such that the MCFA composition may include caprylic acid, capric acid, lauric acid, or combinations thereof. Embodiments may additionally or alternatively exclude one or more other MCFAs, such as heptanoic acid, nonanoic acid, and/or undecanoic acid. Some embodiments may include one or more of caproic acid, caprylic acid, capric acid, and/or lauric acid. Pursuant to such embodiments, the nonanoic acid may be used to supplement or replace one or more MCFAs included in the MCFA composition.

In embodiments featuring two or more MCFAs, the ratio of the MCFAs may vary. For example, embodiments may feature MCFAs provided in approximately equal amounts, or equal concentrations of two MCFAs, and more or less of a third MCFA. In an example embodiment, the MCFA composition consists of caproic acid, caprylic acid, capric acid and lauric acid in approximately equal amounts. In some embodiments, the ratio of MCFAs included in the MCFA composition may not be equal. According to such examples, the ratio of each component in the composition may be different. Specific examples may include approximately equal amounts of caprylic and lauric acid or approximately equal amounts of capric acid and lauric acid. MCFA compositions disclosed herein may contribute to the effective mitigation of PRRS by reducing viral load in serum and/or lung tissue, and/or reducing the number and/or presence of bacterial pathogens in afflicted animals, for example. Where two or more MCFAs are present, embodiments may feature two MCFAs with respect to each other at a ratio of about 1:1, 1:2, 2:3, 1:3, 1:4, 1:5, 1:6, 1:7, 1:8, 1:9, 1:10, 4:5, or any ratio therebetween. Where three or more MCFAs are present, embodiments may feature three MCFAs with respect to each other at a ratio of about 1:1:1, 1:1:2, 1:2:2, 1:2:3, 1:3:3, 1:3:5, 1:2:4, 2:2:3, 2:3:3, 2:3:5, 3:3:5, 3:4:5, 1:4:4, 1:5:5, 1:6:6, 1:7:7, 1:8:8, 1:9:9, 1:10:10 or any ratio therebetween.

Examples of the MCFA composition may include pure fatty acids, not glyceride compositions. Pure fatty acids may be more effective to inhibit PRRSV than mono-, di-, or triglycerides of one or more fatty acids. The pure MCFA(s) may be fully hydrogenated. Embodiments can also include mono-, di-, or triglycerides of one or more of the fatty acids.

The form of the MCFA composition may vary. In embodiments, the MCFA composition may be a dry composition, e.g., a powder or granular substance. The MCFA composition may also be provided as a liquid or mealy paste. Embodiments may also include a dry MCFA composition formulated for mixing with one or more liquids at the time of feeding, for example before admixing the MCFA composition with a feed composition.

The MCFA composition may comprise at least one pure or substantially pure MCFA blended together with one or more additional agents configured to retain the desired properties of the MCFAs and/or improve the palatability of the MCFAs to the target animals. Such agents may include food-safe anti-caking agents, flavorants, desiccants, emulsifiers, preservatives, stabilizers, salts, and combinations thereof.

The concentration of pure or substantially pure MCFAs within the MCFA composition may vary. In embodiments, the pure or substantially pure MCFAs constitute the primary component by dry weight percentage of the MCFA composition. In additional examples, the pure or substantially pure MCFAs may constitute at least up to about 25 wt %, to about 30 wt %, to about 35 wt %, to about 40 wt %, to about 45 wt %, to about 50 wt %, to about 55 wt %, to about 60 wt %, to about 65 wt %, to about 70 wt %, to about 75 wt %, to about 80 wt %, to about 85 wt %, to about 90 wt %, to about 95 wt %, to about 98 wt %, to about 99 wt %, or to about 100 wt % of the MCFA composition.

The MCFA(s) included in the MCFA composition may be derived from corn kernels. Blends of commercially available MCFAs may also be obtained and used to form compositions of one or more MCFAs. In addition or alternatively, the original source of the MCFAs may include hydrolysates of plant oils such as corn, palm, palm kernel oil, coconut, canola etc. One or more synthetic MCFAs may also be included in some embodiments.

In certain embodiments of the feed products, for example embodiments featuring feed products formulated for piglets, recently weaned pigs, growing phase pigs, and/or finishing phase pigs, the MCFA composition may constitute about 20 wt % to about 60 wt % of the feed product, or up to about 25 wt %, to about 30 wt %, to about 35 wt %, to about 40 wt %, about 45 wt %, about 50 wt %, to about 55 wt %, or any value therebetween, including ranges of about 30 wt % to about 50 wt %, about 32 wt % to about 48 wt %, about 34 wt % to about 46 wt %, about 36 wt % to about 44 wt %, about 38 wt % to about 42 wt %, or about 39 wt % to about 41 wt % of the feed product.

The DFM(s) included in the disclosed feed products may exhibit robust production of various enzymes (e.g., amylase, lipase, proteinase, cellulase), strong pathogen inhibition, and are safe for inclusion in animal feeds. A single DFM or a blend of two or more DFMs may be included in the feed products and may be referred to as a “DFM composition” regardless of the number of unique strains included therein. For example, each feed product may include a DFM composition comprised of one or more strains of Bacillus, such as B. pumilus, B. subtilis, and/or B. amyloliquefaciens. Embodiments are not limited to strains of Bacillus, however, and may include additional bacterial strains in the same or different genus. Specific embodiments may include two strains of B. pumilus or a combination of B. amyloliquefaciens and B. subtilis.

In embodiments featuring two or more bacterial strains, the ratio between them may vary. For example, embodiments may feature approximately equal amounts or concentrations of two or more distinct strains of bacteria. Alternative embodiments may feature two or more strains present at a ratio of about 1:2, 2:3, 1:3, 1:4, 1:5, 1:6, 1:7, 1:8, 1:9, 1:10, or any ratio therebetween. The absolute and relative amounts of the DFM composition may vary within different embodiments of the disclosed feed products. In certain embodiments of the feed products, the DFM composition may constitute about 0.1 wt % to about 10.0 wt % of the feed product, or up to about 0.2 wt %, to about 0.3 wt %, to about 0.4 wt %, to about 0.5 wt %, to about 0.6 wt %, to about 0.7 wt %, to about 0.8 wt %, to about 0.9 wt %, to about 1.0 wt %, to about 1.5 wt %, to about 2.0 wt %, to about 2.5 wt %, to about 3.0 wt %, to about 3.5 wt %, to about 4.0 wt %, to about 4.5 wt %, to about 5.0 wt %, to about 5.5 wt %, to about 6.0 wt %, to about 6.5 wt %, to about 7.0 wt %, to about 7.5 wt %, to about 8.0 wt %, to about 8.5 wt %, to about 9.0 wt %, to about 9.5 wt % of the feed product, or any value therebetween, including ranges of about 0.1 wt % to about 0.8 wt %, about 0.15 wt % to about 0.6 wt %, about 0.20 wt % to about 0.4 wt %, about 0.22 wt % to about 0.38 wt %, about 0.22 wt % to about 0.26 wt %, or about 0.30 wt % to about 0.34 wt % of the feed product.

Direct-fed microbial compositions disclosed herein may include pure or substantially pure bacterial isolates, which may include preexisting isolates, novel isolates, or novel isolate combinations discovered during the development of the feed products disclosed herein. The concentration of pure or substantially pure DFMs within a given DFM composition may vary. In embodiments, the pure or substantially pure DFMs constitute the primary component by dry weight percentage of the DFM composition, such that the pure or substantially pure DFMs constitute at least about 50 wt %, about 55 wt %, about 60 wt %, about 65 wt %, about 70 wt %, about 75 wt %, about 80 wt %, about 85 wt %, about 90 wt %, about 95 wt %, about 98 wt %, about 99 wt %, or about 100 wt % of the DFM composition.

The DFM compositions may be provided in media-free form. Metabolites of one or more bacterial strains included in a DFM composition may also be utilized, as can mixtures of live bacteria and their metabolites and/or fermentate. Non-limiting examples of such metabolites can include one or more lipopeptides and/or bacteriocins. Mutant and/or transformed strains of the bacteria disclosed herein may also be used. In some examples, one or more bacterial strains included in a feed product may originate from swine, thereby facilitating colonization of the bacteria in pigs. This feature may be directed to embodiments targeting swine because colonization can enhance the long-term benefits of the strains to the targeted hosts.

The DFM composition may comprise at least one pure or substantially pure DFM blended together with one or more additional agents configured to retain the desired properties of the DFMs and/or improve the palatability of the feed product to the target animals. Such agents may include anti-caking agents, flavorants, desiccants, emulsifiers, preservatives, stabilizers, salts, and combinations thereof.

The flavonoids included in embodiments of the disclosed feed products may be of plant origin, e.g., plant-derived substances, products, extracts, and/or oils. The flavonoid(s) may exhibit antioxidant, anti-inflammation properties. The specific components of the flavonoid composition may vary and may be adjusted depending on several factors, including the respiratory health of the target animals. In embodiments, a flavonoid composition may include one or more additional active components or substances. In various embodiments, non-limiting examples of the active components or substances of the flavonoid composition may include one or more citrus flavonoids (e.g., citrus aurantium), berry flavonoids, and other flavonoids, which may be provided separately or together with one or more phytogenic compounds, such as quillaja extract (e.g., quillaja saponaria), curcuma extract (e.g., Curcuma longa), Capsicum annuum, and/or at least one essential oil such as thymol and/or eugenol. Embodiments may include cayenne pepper derived from the Capsicum annuum species of peppers. The absolute and relative amounts of each component in the flavonoid composition may vary.

In certain embodiments of the feed products, for example embodiments featuring feed products formulated for piglets, recently weaned pigs, growing phase pigs, and/or finishing phase pigs, the flavonoid composition may constitute about 0.5 wt % to about 5.0 wt % of the feed product, or up to about 1.0 wt %, to about 1.5 wt %, to about 2.0 wt %, to about 2.5 wt %, to about 3.0 wt %, to about 3.5 wt %, to about 4.0 wt %, to about 4.5 wt % of the feed product, or any value therebetween, including ranges of about 1 wt % to about 3 wt %, about 1.5 wt % to about 2.5 wt %, or about 1.8 wt % to about 2.2 wt % of the feed product.

The organic acid(s) included in embodiments of the disclosed feed products may provide antimicrobial benefits, and may improve palatability and digestion, thereby causing animals to ingest more of the disclosed feed products that include at least one organic acid. In some embodiments, an organic acid composition may include benzoic acid, which may be included concomitantly with one or more additional organic acids such as fumaric acid, butyric acid, propionic acid, and/or lactic acids.

In certain embodiments of the feed products, for example embodiments featuring feed products formulated for piglets, recently weaned pigs, growing phase pigs, and/or finishing phase pigs, the organic acid composition may constitute only about 0 wt % to about 10.0 wt % of the feed product. In some embodiments, feed products formulated for piglets, recently weaned pigs, growing phase pigs, and/or finishing phase pigs may lack organic acids.

For specific embodiments of the feed products disclosed herein, such as the embodiment featuring the add-pack fed to the nursery pigs included in the below-summarized Trial 1 and the embodiment featuring the test feed product admixed with a basal feed and fed to the wean-to-finish pigs included in Trial 2, the MCFA composition may constitute about 37 wt % to about 45 wt % of the feed product, the DFM composition may constitute about 0.1 wt % to about 0.4 wt % of the feed product, and the flavonoid composition may constitute about 1 wt % to about 4 wt % of the feed product. The remainder of the feed product may comprise one or more carrier components.

Some embodiments of the disclosed feed products may specifically exclude mineral clay, glucan/mannans, and/or silica. One or more of such components may interfere with the optimal composition of a disclosed feed product, thereby inhibiting its effectiveness.

The feed products disclosed herein may inhibit one or more of the aforementioned bacterial pathogens, e.g., Streptococcus suis, Mycoplasma spp., Staphylococcus spp., Pasteurella spp. and one or more viral pathogens, e.g., PRRSV, in animals provided feed or animal feed ingredients containing the feed products. As a result, animals fed the animal feed or animal feed ingredients may be shielded from the contraction or worsening of respiratory conditions caused by such bacteria and/or viruses, thereby reducing the animals' likelihood of developing symptoms of respiratory stress. The inhibition of pathogenic bacteria and viruses may be especially significant in group feed settings, e.g., in an outdoor or enclosed facility, where animals are more likely to spread pathogens through communal feed rations, e.g., rations provided in shared feed troughs, feeders, or other containers. Infected animals may expose the feed to pathogens by coughing, breathing, or otherwise contacting the feed, for example. In such scenarios, the disclosed feed products may reduce the survival of the harmful bacteria and viruses deposited on or in the feed before ingestion by one or more additional, non-infected animals.

The active components of the disclosed feed products, i.e., the MCFA(s), DFM(s), flavonoid(s), and/or organic acid(s), may synergistically inhibit PRRSV and one or more bacterial pathogens associated with PRRS or other respiratory conditions, such S. suis and pneumonia. The active components may also exhibit an additive effect when included together. In some examples, it may be imperative to include one or more MCFAs, DFMs, flavonoids, and organic acids in a feed product to effectively inhibit a respiratory condition or associated symptom. In other examples, only a subset of the active components, i.e., one or more MCFAs or DFM(s), may be sufficient to effectively inhibit a respiratory condition or associated symptom. According to such examples, one or more of the active components may provide an ancillary health benefit to the target animals. Ancillary health benefits may not be directly related to respiratory treatment, but may improve overall animal health, thereby increasing the likelihood of effective respiratory treatment. In some embodiments, the feed product may include only an MCFA composition and a DFM composition, without any flavonoids or without any organic acids.

In some embodiments, the feed products may comprise one or more additional agents configured to preserve or protect the active components, enhance the effectiveness of the active components after ingestion, formulate the active components for a specifically timed release, and/or improve the palatability of the active components to the target animals. In some embodiments, the additional agents may include one or more carrier components formulated to protect the active components from immediate degradation. In addition or alternatively, the carrier component(s) may facilitate targeted digestion of the active components. The carrier components may be provided as a coating around the active components, or may be integrally mixed with the active components. In some examples, the carrier components may include wheat bran and/or calcium carbonate. Non-limiting examples of additional agents may include anti-caking agents, flavorants, desiccants, emulsifiers, preservatives, stabilizers, protective coatings, salts, and combinations thereof. The agents may be food-safe for the selected animal. The absolute and relative amounts of each additional agent may vary in different embodiments.

In some examples, the feed products may be partially or wholly encapsulated or coated for controlled release of one or more active components contained therein. Encapsulation may protect a given feed product from early degradation in animals after ingestion. Early degradation may be defined as degradation and/or digestion upstream of the stage of digestion optimal for nutrient absorption into the animals' bloodstream. Accordingly, the timing and location of early degradation likely varies in different animals. The encapsulant may be pH-activated in some embodiments, such that the encapsulant is formulated to decompose and release the active component(s) in an environment having a specific pH, e.g., the small intestine. In some embodiments, the encapsulant may comprise at least one microbial cell wall-based component. In addition or alternatively, the encapsulant may contain one or more fat- or wax-based components, e.g., fatty acid glycerides, formulated to protect the active component(s) from early degradation, thereby preserving them for maximum absorption. Acceptable examples of fat-based encapsulants are described in U.S. Pat. No. 9,986,749 to Boucher et al., the entire contents of which are incorporated by reference herein. In some examples, the encapsulant may comprise lipid-based nanoparticles (about 200-500 nm in diameter).

The disclosed feed products may be admixed or otherwise combined with a base feed, e.g., a complete feed, feed ingredients, starter feed, a basal feed, a growing phase feed, and/or a finishing phase feed in an amount effective to inhibit a respiratory condition in animals fed the resulting mixture. The form of the base feed may vary. For example, the base feed may be provided as a pellet, meal, or crumble. The base feed may provide the basic nutritional and bulk feed components needed in the diet of the animals. In some examples, the base feed may be the primary food source, by weight, of the animals' diet, which may be supplemented by other natural food sources, e.g., forages. In embodiments, the physical form and/or composition of the base feed may vary. For example, the base feed may be provided as a pellet of variable size, e.g., 3/32″ or 11/64″. In other embodiments, the base feed may include extruded nuggets, granular mixtures, feed blocks, mineral blocks, feed tubs, paste compositions, gels, and/or combinations thereof. Accordingly, the texture and moisture level of the base feed may also vary.

In various embodiments, the feed or feed components may comprise or consist of a complete swine feed, blood meal, porcine meat and bone meal, and spray-dried plasma. The complete swine feed may comprise a feed composition containing all or substantially all the nutritional components a pig needs on a daily basis. Such nutritional components can include one or more sources of protein, e.g., lysine, and one or more carbohydrates including grain, wheat and/or oats. Sources of fiber can also be included, along with various vitamins and minerals, e.g., vitamins A, D and E, copper and/or zinc. Specific embodiments of the feed components may include vitamin D, lysine hydrochloride, choline chloride, and/or soybean meal.

Embodiments of the base feed disclosed herein may comprise a complete animal feed, which may be a grain-based feed formulated specifically for swine, e.g., nursery pigs, weaned pigs, pigs in the growing phase, and/or pigs in the finishing phase. At least a portion of the base feed may comprise corn or corn-based products, wheat or wheat-based products, soybeans or soybean-based products, poultry byproducts, grain sorghum, barley, or combinations thereof. Non-limiting example components of acceptable base feeds can include one or more of: ground corn, sugar, dehulled soymeal, soy isolate, hydrolyzed soy protein, dried distillers grains with solubles, poultry byproduct meal, or any combination thereof, along with various vitamins, minerals, and/or additives, non-limiting examples of which may include: calcium carbonate, choline chloride, lysine, methionine, selenium, threonine, tryptophan, valine, zinc oxide, lactose source, salt, selenium yeast, and/or intake enhancing product.

The nutrient profile of the base feed generally includes various amounts of crude protein, crude fat, crude fiber, carbohydrates, and assorted vitamins and minerals, each combined in various amounts to produce a feed having variable dry matter and moisture content. This disclosure is not limited to any particular nutrient profile, such that the disclosed feed products can be admixed or otherwise combined with base feeds having various amounts of protein, fat, fiber, carbohydrates, sugar, etc. The total protein content of the base feed may be about 14 wt %, about 15 wt %, about 16 wt %, about 17 wt %, about 18 wt %, about 19 wt %, about 20 wt %, about 21 wt %, about 22 wt %, about 23 wt %, about 24 wt %, or more, or any level of protein therebetween. Embodiments of the base feed may contain crude fat at a level ranging from about 1 wt % to about 10 wt %, for example about 2 wt %, about 3 wt %, about 4 wt %, about 5 wt %, about 6 wt %, about 7 wt %, about 8 wt %, or any level therebetween. Embodiments of the base feed may contain fiber at a level ranging from about 1 wt % to about 10 wt %, for example about 2 wt %, about 3 wt %, about 4 wt %, about 5 wt %, about 6 wt %, about 7 wt %, about 8 wt %, about 9 wt %, or any level therebetween. Embodiments of the base feed may contain one or more amino acids, e.g., lysine, at a level ranging from about 0.1 wt % to about 2 wt %, for example about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, or any level therebetween. Additional vitamins and minerals, such as monocalcium phosphate, dicalcium phosphate, calcium carbonate, sodium carbonate, sodium bicarbonate, sodium chloride, potassium chloride, potassium carbonate, potassium iodate, magnesium oxide, ferric oxide, ferrous oxide, calcium oxide, calcium hydroxide, chromic oxide, copper oxide, copper sulfate, zinc oxide, calcium chloride, copper sulfate, trace amounts of selenium, chromium, cobalt, molybdenum, manganese, fluoride, iodine, and the like may be present at various levels.

After combining the feed product with a base feed, the resulting product may be dry or substantially dry, such that moisture level is zero, or close to zero. In some examples, moisture may be present in the base feed at less than about 5 wt %, such as about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.5 wt %, about 2 wt %, about 2.5 wt %, about 3 wt %, about 3.5 wt %, about 4 wt %, about 4.5 wt %, or any level therebetween.

After admixing with a base feed, the disclosed feed products may constitute various levels of the resulting mixture. The feed products, each containing an MCFA composition and a DFM composition, together with one or more of a flavonoid composition and/or organic acids, may be present within a feed mixture at levels ranging from about 0.01 wt % to about 1.0 wt %, including but not limited to about 0.02 wt %, about 0.03 wt %, about 0.04 wt %, about 0.05 wt %, about 0.06 wt %, about 0.07 wt %, about 0.08 wt %, about 0.09 wt %, about 0.1 wt %, about 0.2 wt %, about 0.3 wt %, or about 0.4 wt % of the total feed product.

In embodiments, the feed product may be included in a base feed at inclusion rates of ranging from about 1 lb./ton of feed up to about 50 lbs./ton of feed, or any rate therebetween, such as about 1 lb./ton of feed up to about 1.5 lbs./ton of feed, to about 2.0 lbs./ton of feed, to about 2.5 lbs./ton of feed, to about 3.0 lbs./ton of feed, to about 3.5 lbs./ton of feed, to about 4.0 lbs./ton of feed, to about 4.5 lbs./ton of feed, to about 5.0 lbs./ton of feed, to about 5.5 lbs./ton of feed, to about 6.0 lbs./ton of feed, to about 6.5 lbs./ton of feed, to about 7.0 lbs./ton of feed, to about 7.5 lbs./ton of feed, to about 8.0 lbs./ton of feed, to about 8.5 lbs./ton of feed, to about 9.0 lbs./ton of feed, to about 9.5 lbs./ton of feed, to about 10.0 lbs./ton of feed, to about 10.5 lbs./ton of feed, to about 11.0 lbs./ton of feed, to about 11.5 lbs./ton of feed, to about 12.0 lbs./ton of feed, to about 12.5 lbs./ton of feed, to about 13.0 lbs./ton of feed, to about 13.5 lbs./ton of feed, to about 14.0 lbs./ton of feed, to about 14.5 lbs./ton of feed, to about 15.0 lbs./ton of feed, to about 15.5 lbs./ton of feed, to about 16.0 lbs./ton of feed, to about 16.5 lbs./ton of feed, to about 17.0 lbs./ton of feed, to about 17.5 lbs./ton of feed, to about 18.0 lbs./ton of feed, to about 18.5 lbs./ton of feed, to about 19.0 lbs./ton of feed, to about 19.5 lbs./ton of feed, to about 20.0 lbs./ton of feed, to about 25.0 lbs./ton of feed, to about 30.0 lbs./ton of feed, to about 35.0 lbs./ton of feed, to about 40.0 lbs./ton of feed, to about 45.0 lbs./ton of feed, to about 50.0 lbs./ton of feed, or more.

In embodiments, the DFM compositions of the feed products may be included in a base feed at inclusion rates ranging from less than about 50,000 CFUs/gram to about 100,000 CFUs/gram, about 200,000 CFUs/gram, about 300,000 CFUs/gram, about 400,000 CFUs/gram, about 500,000 CFUs/gram, about 600,000 CFUs/gram, about 700,000 CFUs/gram, about 800,000 CFUs/gram, about 900,000 CFUs/gram, about 1 million CFUs/gram, about 10 million CFUs/gram, about 20 million CFUs/gram, about 30 million CFUs/gram, about 40 million CFUs/gram, about 50 million CFUs/gram, about 60 million CFUs/gram, about 70 million CFUs/gram, about 80 million CFUs/gram, about 90 million CFUs/gram, about 100 million CFUs/gram, about 100 million CFUs/gram to about 400 million CFUs/gram, greater than 400 million CFUs/gram, or any concentration therebetween, such as about 120 million CFUs/gram, about 140 million CFUs/gram, about 160 million CFUs/gram, about 180 million CFUs/gram, about 200 million CFUs/gram, about 220 million CFUs/gram, about 240 million CFUs/gram, about 260 million CFUs/gram, about 280 million CFUs/gram, about 300 million CFUs/gram, about 320 million CFUs/gram, about 340 million CFUs/gram, about 360 million CFUs/gram, or about 380 million CFUs/gram.

In embodiments, the flavonoid composition of the feed products may be included in a base feed at inclusion rates ranging from less than about 20 g/ton to about 20 g/ton, about 20 g/ton to about 500 g/ton, greater than 500 g/ton, or any concentration therebetween, such as about 40 g/ton, about 50 g/ton, about 60 g/ton, about 70 g/ton, about 80 g/ton, about 90 g/ton, about 100 g/ton, about 110 g/ton, about 120 g/ton, about 130 g/ton, about 140 g/ton, about 150 g/ton, about 160 g/ton, about 170 g/ton, about 180 g/ton, about 190 g/ton, about 200 g/ton, about 210 g/ton, about 220 g/ton, about 230 g/ton, about 240 g/ton, about 250 g/ton, about 260 g/ton, about 270 g/ton, about 280 g/ton, about 290 g/ton, about 300 g/ton, about 310 g/ton, about 320 g/ton, about 330 g/ton, about 340 g/ton, about 350 g/ton, about 360 g/ton, about 370 g/ton, about 380 g/ton, about 390 g/ton, about 400 g/ton, about 410 g/ton, about 420 g/ton, about 430 g/ton, about 440 g/ton, about 450 g/ton, about 460 g/ton, about 470 g/ton, about 480 g/ton, or about 490 g/ton.

In some examples, the feed products may be formulated for admixing with a base liquid, such as water or milk replacer, in an amount effective to inhibit PRRS in animals fed the resulting solution, suspension or mixture, either alone or in combination with a feed composition supplemented with the feed products. The feed products may also be formulated for admixing with a base liquid, such as water or milk replacer, in an amount effective to inhibit a condition caused by one or more bacterial pathogens, such as S. suis and pneumonia, in animals fed the resulting solution, suspension or mixture, either alone or in combination with a feed composition supplemented with the feed products. The components and nutritional profiles of such liquids may vary, and may depend on the targeted animals.

The fat content of milk replacers into which the feed products may be mixed may range from about 2.25 wt % to about 4.7 wt % of the hydrated milk replacer or from about 8 wt % to about 31 wt % of the milk replacer powder. Protein in milk replacers typically ranges from about 2.2 wt % to about 5.1 wt % of the hydrated milk replacer or about 18 wt % to about 30 wt % of the powder. The protein content may be about 22 wt % of the powder or about 3.3 wt % of the rehydrated milk replacer. Protein may be sourced from animal (e.g., milk, plasma, egg, and red blood cells) and vegetable sources and combinations thereof. Milk-derived protein sources are generally referred to as milk proteins and may include whey, casein, skim milk, sodium caseinate, and calcium caseinate. Non-milk proteins (NMPs), such as vegetable protein (e.g., soy protein, hydrolyzed soy protein, hydrolyzed soy protein modified, soy protein isolate, wheat concentrates, wheat isolates, pea concentrates, pea isolates, and/or potato proteins), animal protein (e.g., plasma such as bovine or porcine plasma, egg and red blood cells), and single cell protein, alone or in combination, may be included as a protein source in the milk replacer.

In some embodiments, a disclosed feed product can be offered concurrently with or in addition to one or more antibiotics or medicinal compounds. In such embodiments, the feed product and the antibiotic(s) may provide a synergistic or additive effect with respect to respiratory condition inhibition, thereby reducing, eliminating or preventing symptoms of the condition to a greater extent than either the feed product or the antibiotic(s), alone. The amount of antibiotics used in combination with the feed products may be reduced relative to approaches where antibiotics are used without the feed products of the present disclosure. Examples may also exclude one or more antibiotics, and may be antibiotic-free.

### Methods of Mitigating PRRS and Other Pathogenic Conditions

Methods of inhibiting respiratory conditions in animals involve feeding the animals an effective amount of a feed product disclosed herein, either as a stand-alone product or as a component of a base feed or liquid. Non-limiting examples of particular animals fed the disclosed feed products can include various breeds of swine. The animals may have observable respiratory distress or be diagnosed with a respiratory condition, exhibiting symptoms of a respiratory condition, or at risk of contracting a respiratory condition, although methods may also be applied to healthy animals that may not be at a notable risk of contracting a respiratory condition. The respiratory condition may be PRRS alone, or PRRS together with one or more additional respiratory conditions, such as pneumonia, which may be caused in part by a Streptococcus suis infection.

Methods may involve initially forming a feed product or simply obtaining it, for example via purchase. As noted above, embodiments may involve combining the feed product with a base feed or liquid at or prior to feeding. Pursuant to such embodiments, an animal producer may purchase, produce or otherwise obtain a base feed lacking a feed product disclosed herein. Examples can involve admixing the feed product with the base feed on a routine or semi-routine basis, for example once per day, and subsequently offering the resulting mixture to the target animals, for example on the day of mixing or the day after mixing. Embodiments may also involve admixing the feed product with a base feed and placing the resulting mixture in a self-feeder, which may be configured to supply the target animals with adequate amounts of feed for one or more days or weeks at a time, thereby necessitating less frequent admixing of the feed product with the base feed. The feed product can be admixed with the base feed in any convenient manner that ensures the feed product is ultimately ingested by the target animals. For example, the feed product may be admixed with feed components during production of a feed mixture, such as a feed supplement or a premix. The feed product may additionally or alternatively be top-dressed over a base feed or feed components, such top-dressing over feed contained within a feeding trough or deposited on a flat surface. In some embodiments, the base feed may contain a disclosed feed product, such that the animal producer may simply obtain the final feed product and then provide it directly to the animals. The feed product can be fed in any form which is suitable for feeding the animals.

In some examples, the base feeds/liquids and the feed product may be provided to end users in separate containers or packages. In such embodiments, the user may be instructed to admix the feed product with the base feed and/or liquid according to one or more instructions provided on the packaging for the feed product. Instructions may indicate acceptable amounts of feed product to be admixed with various amounts of feed and/or liquid. Instructions may also provide methods of adjusting the concentration of the feed product within the base feed and/or liquid in response to variations in the severity of respiratory stress exhibited by the animals. The instructions may also indicate acceptable time frames to begin feeding animals in anticipation of conditions conducive to respiratory stress. In some embodiments, the instructions may include one or more warning signs, e.g., symptoms of respiratory stress, prompting end users to begin mixing the feed product with the base feed or liquid and providing the resulting composition to the animals.

According to certain implementations of use, a disclosed feed product may be provided in the diet of the animal during all or a portion of the year in which the animal is or may be challenged by respiratory-associated issues (e.g., based on historical seasonal patterns of respiratory sickness) to improve animal performance during such periods of stress. For instance, the feed product may be provided during the seasonal changes, such as fall to winter, and winter to spring, or during the winter months. The feed product may also be provided intermittently during the year in anticipation of periods of respiratory-associated issues (e.g., where an animal is housed with other animals showing signs or symptoms of respiratory-associated issues), and for instance the animal may be prophylactically provided the product.

In another implementation, the feed product may be included in the diet of the animal only during periods of observed respiratory-associated issues in the animal. Respiratory-associated issues may include but are not limited to infections affecting the lungs of the animal, for instance caused by pathogens responsible for respiratory-associated diseases including PRRSV, Streptococcus, Staphylococcus, Bordetella, Pasteurella and other pathogens, respiratory stress, increased respiration from about 45-75 cycles per minute standing, or about 40-70 cycles per minute recumbent, panting, shallow breathing, coughing and combinations of such respiratory-associated issues. Respiratory-associated issues may also decrease animal performance (e.g., decreased rate of weight gain, increase weight loss, and decrease feed or fluid intake (e.g., reductions of about 5%, 10%, 15%, 20%, or more)), and/or increase other symptoms such as elevated internal body temperature, open mouth breathing, excessive panting, sweating, lethargy, shakiness, thumping, back end weakness, increased spine curvature, lameness, and failed reproduction may also be attributed to respiratory-associated issues experienced by the animal, for example those caused by PRRSV and/or Streptococcus infections, e.g., Streptococcus suis infections. Three different presentations of Streptococcus may appear: (1) peracute, (2) acute, and (3) chronic. The peracute form may not present any clinical signs; the animals may simply be found dead without warning. For the acute form, an initial symptom may be fever and associated anorexia and depressed behavior. Affected animals often present signs of meningitis, such as squinting eyes and flattened ears. Depression, paddling, ataxia, nystagmus, and head tilts are also common, especially for infections caused by Streptococcus suis. Polyarthritis is a common symptom of the chronic form and is frequently accompanied by swollen joints and lameness. Streptococcus suis may cause or contribute to cases of pneumonia, and/or may be a secondary and opportunistic pathogenic bacteria associated with the condition.

The disclosed methods may be implemented on pig farms, where the pigs may be raised and maintained indoors and/or outdoors. The disclosed methods may also be beneficial for pigs particularly susceptible to viral and/or bacterial infection. Susceptibility to infection may be greater for pigs raised in certain areas under conditions conducive to viral transmission. Susceptibility to infection may also be greater for pigs suffering from one or more medical ailments, pigs raised in close proximity to large numbers of other pigs, or younger pigs with undeveloped immune systems. In some embodiments, the disclosed methods may involve feeding a disclosed feed product to animals that are afflicted with or at risk of contracting a pathogenic infection, but are otherwise healthy, e.g., not heat-stressed.

Particular embodiments may involve feeding the animals a disclosed feed product over a feeding period when the animals are typically confined to an indoor facility or an outdoor area and placed under tightly controlled conditions. For example, access to feed and/or water may be restricted by animal caretakers within the indoor facility or outdoor area, such that the animals are not free to consume as much of the feed and/or water as they would outside the facility or elsewhere. The feeding period can encompass one or more discrete developmental and/or reproductive stages, e.g., pre-weaning, weaning, and post-weaning (e.g., nursery pigs) and/or any transitional phases therebetween. The feeding period can also encompass all or a portion of a growing phase and a finishing phase, which may involve admixing a disclosed feed product with one or more different base feed compositions.

The disclosed feed products may be fed to animals of any age for any length of time. The length of the feeding period may vary for different animals, or it may be approximately the same, again depending on a multitude of factors which may include the age of the animals and whether the animals are infected or at risk of becoming infected with PRRSV and one or more bacterial pathogens associated therewith. In embodiments, the feed product may be fed over a feeding period spanning about one week or less, or longer than one week, for e.g., two weeks, three weeks, four weeks, five weeks, six weeks, seven weeks, eight weeks, nine weeks, ten weeks or more, for example periods lasting from about one month to about five months, six months, eight months, ten months, one year, two years, three years, four years, five years, six years, seven years, eight years, or more, or any length of time therebetween. In embodiments, the animals may be provided the feed product for as long as the PRRSV and/or S. suis is detectable within the animals, for as long as the animals exhibit symptoms of PRRS and/or pneumonia, or until the animals are slaughtered. In some embodiments, one or more animals may be fed the feed product for a period of time after the disappearance of respiratory stress indicators to prevent a reemergence of one or more respiratory conditions. Animals may be fed the feed products before, during, and/or after weaning, and may be admixed with different feed or liquid compositions as a result. For example pigs may ingest the feed products via a pelleted feed and/or meal composition before, during, and/or after weaning. The animals may be variously sized during the feeding period(s). For example, swine offered the disclosed feed products may range from about 10 pounds to about 200 pounds or more, or any weight therebetween. In some examples, young pigs fed the disclosed feed products can include piglets or nursery pigs about 0, 7, about 15, about 21, about 28, about 35 or more days after weaning. The young pigs may be fed for about 14, about 21, about 28, about 35, about 42, or more days, or about 14 to about 35 days, or at least 14, at least 21, at least 28 or at least 35 days. Embodiments may also involve feeding weaned pigs for a post-weaning period lasting at least about 1 week, 2 weeks, 3 weeks, 4 weeks, 5 weeks, 6 weeks, 7 weeks, 8 weeks, 9 weeks, 10 weeks or longer. The pigs, including young pigs, weaned pigs, growing pigs, and finishing pigs, may be fed in a group setting or individually.

Embodiments may involve feeding the animals a disclosed feed product on an ad libitum basis throughout a feeding period. An ad libitum basis as contemplated herein means that feed product is placed in a feeder or on a suitable surface (for example within a broader feed or liquid composition) and the animal consumes it until satisfied, such that the feed products described herein are always available to the animal for consumption at free will. In additional embodiments, one or more animals may be fed according to a more controlled feeding regimen, such that one or more defined rations are offered one or more times per day, e.g., once daily, twice daily or three times daily. The particular manner in which a feed product is fed to the animals is not intended to be limited. Any manner suitable for feeding animals may be satisfactory.

The amount of the feed product ingested per animal may vary and may depend on a numerous factors, non-limiting examples of which include the starting concentration of one or more components of the feed product, the number of feedings administered per day, the specific breed or species of the target animal, the age and/or size of the target animal, the life stage of the target animal, the concentration of the feed product within the final feed or liquid composition, and/or whether the target animal has been diagnosed with or is exhibiting symptoms of a respiratory condition. In some embodiments, each target animal may ingest less than or up to about 1 gram of the feed product per day, or up to about 2 grams per day, to about 3 grams per day, to about 4 grams per day, to about 5 grams per day, to about 6 grams per day, to about 7 grams per day, to about 8 grams per day, to about 9 grams per day, to about 10 grams per day, to about 20 grams per day, to about 30 grams per day, to about 40 grams per day, to about 50 grams per day, to about 60 grams per day, to about 70 grams per day, to about 80 grams per day, to about 90 grams per day, to about 100 grams per day, to about 125 grams per day, to about 150 grams per day, to about 175 grams per day, to about 200 grams per day, to about 225 grams per day, or more, or any amount therebetween. In specific embodiments, animals from about 18 to about 22 days of age offered ad libitum access to a complete feed containing a disclosed feed product at an inclusion rate of about 10 lbs./per ton of feed may consume about 2.54 grams of the feed product per head per day, or about 0.5 to about 5.0 grams per head per day, or up to about 1.0 grams per head per day, to about 1.5 grams per head per day, to about 2.0 grams per head per day, to about 2.5 grams per head per day, to about 3.0 grams per head per day, to about 3.5 grams per head per day, to about 4.0 grams per head per day, to about 4.5 grams per head per day, or more, or any rate therebetween. The amount or concentration of the feed products provided to the animals can remain substantially constant or change over time, for example such that the daily feeding rate and/or inclusion rate may be different for pre-weaned or recently weaned animals relative to partially- or fully-grown animals, e.g., swine in the lactation, growing, or finishing phase. For instance, animals having a body weight ranging from about 15 to about 25 lbs. may be offered the a complete feed containing a disclosed feed product at an inclusion rate of about 6 lbs. to about 10 lbs. per ton of the resulting total feed, about 7 lbs. to about 9 lbs. per ton of total feed, about 8 lbs. per ton of total feed, or more, or any amount therebetween. Animals having a body weight ranging from about 25 to about 150 lbs. may be offered the feed product at an inclusion rate of about 1 lb. to about 7 lbs. per ton of total feed, about 2 lbs. to about 6 lbs. per ton of total feed, about 3 lbs. to about 5 lbs. per ton of total feed, or about 4 lbs. per ton of total feed, or any amount therebetween. Animals having a body weight ranging from about 150 lbs. to about market weight may be offered the feed product at an inclusion rate of about 0.5 lbs. to about 4 lbs. per ton of total feed, about 1 lb. to about 3 lbs. per ton of total feed, or about 2 lbs. per ton of total feed, or any amount therebetween.

The amount of base feed or liquid ingested per animal per day can vary, and may depend largely on the age and breed of the animal. Swine ingesting a pelleted feed composition containing the feed product, for example, may ingest about 0.1 lbs. to about 10 lbs. per day, about 0.5 lbs. to about 8 lbs. per day, about 1.0 lbs. to about 6 lbs. per day, about 2 lbs. to about 4 lbs. per day, about 3 lbs. to about 4 lbs. each day, greater than 10 lbs. per day, or any amount therebetween. Piglets, may consume an average of about 1.2 lbs. to about 1.3 lbs. of feed per day for about the first 6 weeks after weaning. Swine ingesting a meal feed composition containing the feed product may consume the same or similar amounts of feed on a daily basis. The embodiments disclosed herein are not limited to any particular daily consumption rate.

The disclosed embodiments are not limited to one mechanism or theory of inhibition; however, feeding the disclosed feed products to animals can trigger the gut-lung immune axis and improve the overall health of the animal recipients. Through competitive exclusion and the production of antimicrobial substances, for instance, the DFM compositions included in the disclosed feed products may function as probiotics configured to inhibit the growth of multiple pathogens, including gram positive and gram negative bacteria, which frequently contribute to the development of respiratory conditions. Specific embodiments may exhibit an antibacterial effect against strains of Streptococcus suis. The feed products may also ameliorate one or more effects underlying or exacerbating a respiratory condition. For example, ingestion of the feed products may balance pro-inflammation and anti-inflammation, boost antioxidation, improve gut barrier function, improve nutrient absorption and transport, stimulate endogenous enzyme production, and/or enhance the antimicrobial quotient of the target animals' microbiome. As noted above, the feed products disclosed herein may become active after ingestion by the target animals, for example upon reaching the animals' GI tract, where the feed products may be activated in the manner necessary to release bacteriocins specific to one or more pathogenic bacterial species or strains implicated in respiratory stress.

Implementation of the methods disclosed herein may improve the performance of animals at risk of contracting, already afflicted with, or recovering from a respiratory condition, such as PRRS. Indicators of improved performance may vary from animal to animal, and may depend on various factors including animal age and/or the severity of respiratory symptoms exhibited by the animal prior to administration of a feed product disclosed herein. In embodiments, improved performance may relate to a direct reversal of one or more respiratory stress symptoms and/or improvements to animal health not directly tied to a reduction in respiratory stress.

In some examples, improved performance may encompass a reduced mortality and removal rate and/or a reduced need for respiratory treatment, for example involving injection or administration of a medication or antibiotic. Disclosed methods may improve animal performance by inhibiting PRRSV and one or more bacterial pathogens, which may prevent or reduce the severity of PRRS and other respiratory conditions caused by viral and/or bacterial pathogens. For pigs already infected with PRRSV and potentially one or more bacterial pathogens such as S. suis, one or more symptoms of PRRS may be alleviated or eliminated by implementing the methods disclosed herein. Embodiments may reduce the incidence and severity of pneumonia evidenced for example by a reduction in pneumonia lesions on the lungs of infected animals. Embodiments may also reduce the level respiratory distress and PRRS viral loads in both serum and lung tissue of PRRSV-infected and bacterially infected animals relative to infected animals not fed a disclosed feed product. Embodiments may reduce secondary nasal pathogens during and after PRRSV infection relative to infected animals not fed a disclosed feed product. Embodiments may maintain nasal and cecal beneficial bacteria and microbial balance during and after PRRSV infection. Additional non-limiting examples of improved performance may include reduced feed-to-gain ratios (e.g., about a 20% reduction or greater), increased average daily weight gain (e.g., about 14% increase or greater), increased average daily feed intake (e.g., about 14% increase or greater) and/or increased total weight gain over various periods of time, increased feed consumption, or combinations thereof, relative to animals infected with PRRSV and potentially one or more bacterial pathogens, e.g., S. suis, fed the same base feed but lacking the feed products disclosed herein.

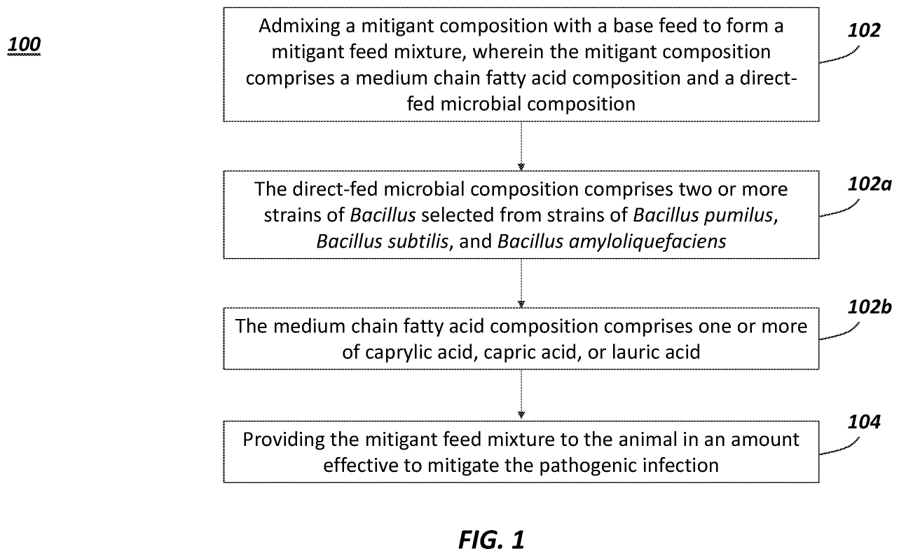

FIG. 1 is a flow diagram of a method of mitigating a pathogenic infection in an animal in accordance with principles of the present disclosure. The example method 100 shows the steps that may be implemented, in any sequence, to mitigate a pathogenic infection by including a mitigant composition in a base feed and providing the resulting mitigant feed mixture to an animal, such as a pig. In additional examples, one or more of the steps shown in the method 100 may be supplemented or omitted. At step 102, the method involves “admixing a mitigant composition with a base feed to form a mitigant feed mixture, wherein the mitigant composition comprises a medium chain fatty acid composition and a direct-fed microbial composition.” As further shown in box 102a, “the direct-fed microbial composition comprises two or more strains of Bacillus selected from strains of Bacillus pumilus, Bacillus subtilis, and Bacillus amyloliquefaciens,” and as further shown in box 102b, “the medium chain fatty acid composition comprises one or more of caprylic acid, capric acid, or lauric acid.” At step 104, the method involves “providing the mitigant feed mixture to the animal in an amount effective to mitigate the pathogenic infection.”

The following examples set forth the effectiveness of various compositions on mitigating PRRS and bacterial infections in pigs. It is to be understood, however, that these examples are provided by way of illustration only, and nothing therein should be viewed as limiting. Numerous modifications and variations are within the scope of the present disclosure as will be apparent to those skilled in the art.

### EXAMPLES

Trial 1

The objective of Trial 1 was to develop and quantify a dual PRRS-S. suis challenge model in nursery pigs and evaluate the impact of feeding pigs co-infected with PRRS and S. suis a feed product disclosed herein.

Materials and Methods Overview. For Trial 1, 26 pigs between the ages of 18-22 days were randomly assigned to three treatment groups: 1) non-challenged negative controls; 2) PRRS and S. suis challenged only; and 3) PRRS and S. suis challenged and fed a disclosed feed product in the form of an “add-pack” comprising an MCFA composition, a DFM composition, and a flavonoid composition.

The experimental add-pack fed to the Group 3 pigs was admixed with a piglet feed at an inclusion rate of about 10 lbs./ton of feed. Average daily consumption of the resulting feed was about 1.15 lbs. per animal per day, such that at least about 2 to about 4 grams of the add-pack were ingested by each animal per day for the 29-day duration of the trial.

Pigs were acclimated for 8 days and challenged with PRRS 1-7-4 isolate on day 8, which corresponded to study day 0 (“SD-0”). On SD-5, Groups 2 and 3 were additionally challenged with S. suis Serotype ½ ST 28. Pigs were monitored twice daily with sample collection and body temperature measurements occurred on study days 0, 5, 10, 14, and 21. All pigs remaining within the treatment groups were humanely euthanized and necropsied on study day 21. The trial began on Feb. 24, 2022 and ended on Mar. 25, 2022.

The primary efficacy criteria analyzed included PRRS viral load in serum and lung tissues, in addition to S. suis semi-quantification in brain, lung and liver tissues. The secondary efficacy criteria included clinical observations, PRRS-specific gross and microscopic lesions and S. suis-specific gross and microscopic lesions in respective groups.

Materials and Methods Detail. During the acclimation period and throughout the remainder of the 29-day trial (21 days post-acclimation), only Group 3 was administered the add-pack via feed, as an additive included in an age-appropriate ration. Groups 1 and 2 did not receive the add-pack and were fed ad libitum an age-appropriate ration for the acclimation and study phase.

The test animals included 26 pigs of mixed gender, age 18-22 days. The animals were bred from a large white, Landrace cross-breed pig from a high health swine multiplication herd in Central, MN. The pigs did not have clinical history and had not been vaccinated for PRRS or S. suis Serotype ½ ST 28. The source farm was screened prior to shipment and pigs were tested for PRRS within 48 hours of arrival by oral fluid collection and PCR testing for confirmation. Tonsil scrapings were collected to screen for S. suis. At arrival, prior to oral fluid collection, pigs were blocked by gender and weight to be placed into test groups. Six selected pigs (Group 1) were moved and housed in a separate airspace. The remaining 20 pigs were allocated into two groups of 10 (Groups 2 and 3) and placed into separate pens with no direct contact.

The treatment and challenge groups were organized in the manner shown below in Table 1.

TABLE 1

| No. of | | | | Dietary |
|---|---|---|---|---|
| Animals | Challenge | Dose | Route | Treatment |
| Group 1 (6 | Sterile MEM | 4 ml/4 | IN (2 ml per | None Diet A |
| head) | | ml | nare)/IN (2 ml | (RED) |
| | | | per nare) | |
| Group 2 (10 | PRRS NA 174/ | 4 ml/4 | IN (2 ml per | None Diet A |
| head) | S. suis | ml | nare)/IN (2 ml | (RED) |
| | Serotype ½ ST 28 | | per nare) | |
| Group 3 (10 | PRRS NA 174/ | 4 ml/4 | IN (2 ml per | Add-pack via |
| head) | S. suis | ml | nare)/IN (2 ml | Feed Diet B |
| | Serotype ½ ST 28 | | per nare) | (GOLD) |

As shown, a total of 26, 18-22-day old iso-wean (isolated and weaning) pigs confirmed to be free of PRRSV, PCV2, SIAV, and S. suis Serotype ½ ST28 were weighed and assigned to one of the three treatment groups upon arrival (SD-0): Group 1, Control group (no challenge and no treatment, 6 head); Group 2, Positive Control group (challenge and no treatment, 10 head); Group 3, Test Group (challenge and add-pack treatment, 10 head). Groups 2 and 3 were housed in the same airspace with no direct contact, while Group 1 was housed in a separate airspace.

During acclimation and throughout the remainder of the trial, all pigs were provided ad-libitum access to feed (Diet A-RED for Groups 1 and 2; Diet B-GOLD for Group 3) and water. Diet A and Diet B were bagged separately, clearly labeled with colored tape and stored in designated bins and locations to ensure diets were fed to the correct group. All totes, scoops and equipment were dedicated and labeled by diet to avoid any cross exposure of the diets. Total feed consumption throughout the trial, for each pen, was measured (pounds of fed feed). Pounds of feed added to the feeder were recorded, feed remaining the next day weighed, and feed added were weighed to track total pounds consumed by pen. Average intake per pig was then calculated by dividing daily pounds fed by the number of pigs in the pen. Initial body weights were measured upon arrival to evenly allocate pigs to treatment groups. Weights were collected on SD 0 (PRRS Challenge) and necropsy days 10 and 21. Any unexpected mortalities were weighed and necropsied at the time of death.

After the acclimation period, Group 1 was sham-inoculated intranasally with 2 ml/nare (4 ml total) of sterile Minimum Essential Medium (MEM). Pigs in Groups 2 and 3 were intranasally inoculated with 2 ml/nare (4 ml total) of diluted NA PPRS 174 challenge strain grown on primary Pulmonary Alveolar Macrophage (PAM) cells and diluted for a total dose of approximately 1×10⁶ log 10 TCID₅₀ per animal using a syringe fitted with an atomizing tip. During challenge the piglets were adequately restrained, and challenge dispensed in one nare at a time gradually upon inhalation. Each piglet remained restrained with the snout elevated to insure challenge inhalation.

At SD-5, Group 1 was sham-inoculated intranasally a second time with 2 ml/nare (4 ml total) of sterile Todd-Hewitt broth with 5% fetal calf serum. Pigs in Group 2 and 3 were additionally inoculated with 4 ml total (2 ml/nare) of S. suis serotype ½ ST28, which were inoculated 5 days earlier with NA PRRS 174 challenge strain.

Oral fluid samples were collected from each pen to assess PRRS exposure on SD-3. All samples were tested via PRRS PCR with a cycle time reported. It was expected that there would be variability in clinical signs between the groups and pigs given that an individual pig's unique response to PRRS virus can be different. Group 1 was expected to have PRRS virus negative oral fluid testing results, as Group 1 was the non-challenged group. Oral fluid samples from Group 2 and 3 were expected to be positive given they were challenged. The clinical presentation of the groups on SD-3 and SD-4 were observed and communicated.

All pigs had their basal body temperature measured on study days 0, 5, 10, 14 and 21 (degrees Fahrenheit). Serum samples were collected from all pigs on study days 0, 5, 10, 14 and 21. Three pigs from Group 1 were selected for necropsy on SD-10. Five pigs from Groups 2 and 3 were necropsied on SD-10. The remaining pigs from each group (Group 1=3, Groups 2 and 3=5) were necropsied on SD-21, or before, if they became moribund. All pigs found deceased on non-necropsy days were necropsied within 12 hours of mortality notice. Nasal swabs were collected from all pigs on SD-10 and the remaining 13 pigs on SD-21. In particular, sterile swabs were taken from both nares of each animal and immediately frozen at −80° C.

For randomizing, treatment groups were blocked by weight and gender with pigs allocated upon arrival. All pigs were weighed and corresponding values recorded in pounds.

Pre-challenge procedures involved collecting total oral fluid samples from the 26 received pigs, one sample per treatment group. Cotton filament ropes were hung at pig height on the pen walls for the pigs to chew on 48 hours after arrival to the facility. Pigs were given access to the ropes for 20-30 minutes in order to saturate the rope with saliva. Ropes were collected in plastic bags, wrung into falcon tubes, and submitted for PRRSV PCR testing. After pigs were allocated to a group, two randomly selected pigs from each group (6 total) were tonsil-swabbed for S. suis screening.

All pigs were observed for general health daily from arrival until challenge. Rectal temperatures were taken from all pigs on study days 0, 5, 10, 14 and 21. Only healthy animals were used in the study with the exception that pigs in Groups 2 and 3 were expected to have varying clinical signs of PRRS at the time of S. suis challenge. Any pigs that were not clinically healthy, as determined by a veterinarian at the time of challenge, were not challenged and ultimately withdrawn.

For challenging, North American Porcine Reproductive and Respiratory Syndrome (NA PRRS) Virus 1-7-4 was administered intranasally, 2 ml/nare (4 ml total). NA PRRS 1-7-4 challenge strain was grown on Pulmonary Alveolar Macrophage (PAM) cells and diluted for a total dose of approximately 1×10⁶ log₁₀ TCID₅₀ per animal using a syringe fitted with an atomizing tip. Streptococcus suis Serotype ½ ST28 was administered intranasally, 2 ml/nare (4 ml total). S. suis Serotype ½ ST28 was acquired from Iowa State University and positive for mrp virulence gene by Next Genome Sequence (NGS) testing. This isolate was harvested from a clinical pig submitted to ISU Diagnostic Laboratory with a PRRS-S. suis co-infection. The S. suis isolate was stabilized with Todd-Hewitt broth with 5% fetal calf serum. This mixture was a total dose of approximately 1×10⁹ colony forming units (cfu)/4 ml dose. Group 1 Controls; First sham-inoculation Minimal Essential Medium, sterile administered intranasally, 2 ml/nare (4 ml total). Second sham-inoculation, Todd Hewitt broth with 5% fetal calf serum sterile administered intranasally, 2 ml/nare (4 ml total).

Challenge administration involved adequately restraining the pigs and dispensing PRRSV/S. suis challenge material or MEM one nare at a time, gradually, upon inhalation. Each piglet remained restrained with the snout elevated to ensure challenge inhalation. Group 1 was sham-inoculated twice, at the time of PRRS and S. suis challenge to Groups 2 and 3. Three replicates of the diluted challenge for both the PRRS and S. suis isolates were confirmed for titer via virus isolation and back titration on sheep blood agar plates.

After challenge, all pigs were observed twice daily beginning one day post-challenge (SD-1) through 21 days post challenge. Rectal temperatures were measured and recorded on study days 0, 5, 10, 14 and 21. Clinical scoring was done in a blinded manner by a team of two people. Clinical observations for respiratory disease, CNS signs, swollen joints, lameness, and diarrhea were recorded and scored based on severity.

Primary variables included the following objective measures between Groups 1, 2 and 3: morbidity, mortality from PRRS and/or S. suis; clinical respiratory score; CNS signs score; joint swelling; lameness score; diarrhea score; brain; liver and lung culture for S. suis (S. suis-positive tissues were semi-quantitative tested for bacterial levels); rectal temperature; serum PRRS viral load; lung tissue PRRS viral load (PCR); lung score/macroscopic interstitial pneumonia for PRRS; macroscopic score for pleuritis, pericarditis, peritonitis, synovial fluid, and meninges for S. suis; microscopic interstitial pneumonia score for PRRS; microscopic lesion score for pleura/pulmonary parenchyma, liver, synovial membrane/tendon sheath/periarticular soft tissue support structures, leptomeninges, and neuroparenchyma for S. suis; initial, challenge and final pig weights; and feed consumption as determined by pounds of feed fed.

Infection with PRRS, S. suis, or a combination of both pathogens resulted in increased mortality/morbidity with interstitial and parenchymal pneumonia, viremia, and septicemia in various forms (meningitis, arthritis, pleuritis, pericarditis, and peritonitis among others). Tissue samples collected for culture and/or PCR testing included brain, liver, lung and clinical joint tissues. Cecum digesta were also collected at necropsy by identifying the ileocaecal junction, cutting open the cecum, and using a spatula to scoop 1-1.5 ml cecal content to one 2-ml tube. The samples were then immediately frozen and stored at −80° C.

PRRS-Specific Necropsy evaluation involved Necropsy, Gross Lesion Scoring, and Tissue Collection: For determining lung score, macroscopic PRRS interstitial pneumonia were scored. Each lung lobe was assigned a number to reflect the approximate volume percentage of the entire lung represented by that lobe. Gross lung lesion scores were estimated and a score assigned to reflect the amount of pneumonia in each lobe. The total points for all the lobes were an estimate of the percentage of the entire lung with grossly visible pneumonia. Sections were taken from all PRRS lesions for formalin fixation. In the absence of PRRS lesions, a section was taken from the right caudal lobe.

PRRS-Specific Lung Lesions: Tissue sections were interpreted for alterations in tissue architecture indicative of interstitial pneumonia-lymphomononuclear cells thickening alveolar septa; Type II pneumocyte hypertrophy and hyperplasia; normal and necrotic alveolar histiocytes plugging alveolar lumina and peribronchiolar/perivascular lymphoplasmacytic cuffs (0=normal; 1=mild focal interstitial pneumonia; 2=moderate multifocal interstitial pneumonia; 3=severe diffuse interstitial pneumonia).

Daily feed consumption was measured in pounds per pen. All feed added or remaining in the feeders was weighed to calculate daily pounds consumed by pen. Average consumption per pig was calculated by dividing daily pounds consumed by number of pigs in the pen.

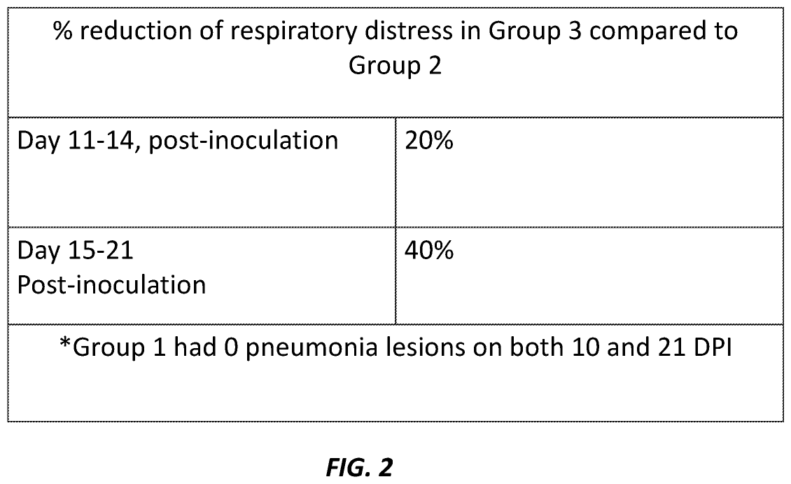

Results. As shown in FIG. 2, consumption of the add-pack significantly reduced the percentage of pigs with respiratory distress. About 40% of Group 2 pigs exhibited respiratory distress at 11-14 days post-inoculation (“DPI”), whereas only about 20% of Group 3 pigs exhibited respiratory distress during the same period. By 15-21 DPI, the difference was even greater, with 100% of Group 2 pigs exhibiting respiratory distress compared to only 60% of Group 3. Accordingly, the Group 3 pigs exhibited a 20% reduction in respiratory distress relative to the Group 2 pigs at 11-14 DPI, and a 40% reduction at 15-21 DPI. The non-challenged control pigs of Group 1 did not exhibit any signs of respiratory distress at both 10 and 21 DPI.

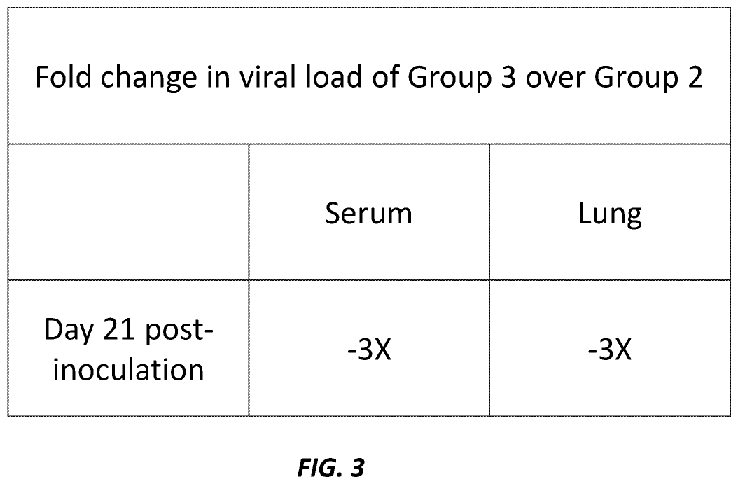

As shown in FIG. 3, the add-pack cleared viremia significantly faster in the serum of the Group 2 pigs by 21 DPI. In particular, the number of viral RNA copies per mL of serum sample was about 1.24×10⁸ for the Group 2 pigs and only 3.82×10⁷ for the Group 3 pigs, marking a 3× reduction in viremia. FIG. 3 shows similar results in lung tissue obtained from the animals, as the number of viral RNA copies per mL of lung tissue was about 2.3×10⁸ for the Group 2 pigs and only 7.25×10⁷ for the Group 3 pigs, marking a 3× reduction in viremia. Lower viremia reflects the development of neutralizing antibodies, indicating the development of immune system in pigs provided the add-pack. Lower viral load in serum and lung samples also indicates a reduction in bacterial pathogens in the animals' microbiome.

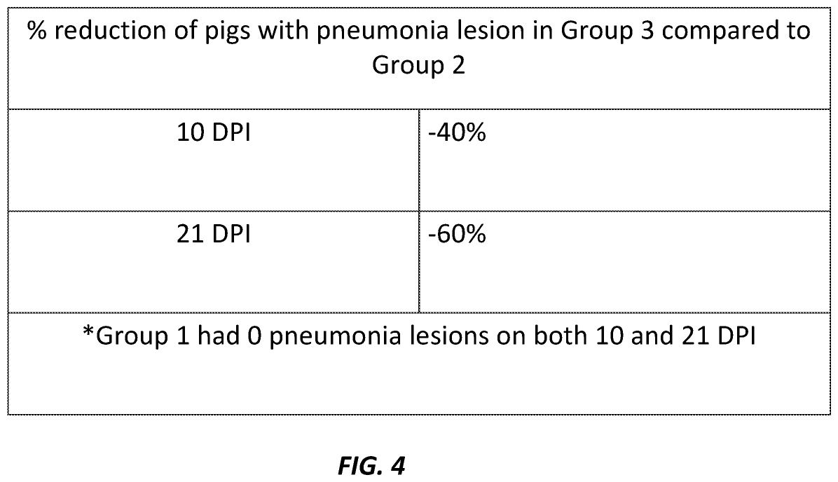

As shown in FIG. 4, consumption of the add-pack significantly reduced the incidence and severity of pneumonia. By 10 DPI, 40% of pigs in Group 2 had at least one pneumonia lesion, whereas none of the Group 3 pigs had a pneumonia lesion. A similar result was observed at 21 DPI, with 80% of Group 2 pigs and only 20% of Group 3 pigs having at least one pneumonia lesion, respectively. Accordingly, the number of pigs having at least one pneumonia lesion after 10 DPI was 40% less than the number of Group 2 pigs at 10 DPI. By 21 DPI, the number of Group 3 pigs having at least one pneumonia lesion was 60% less than the number of Group 2 pigs.

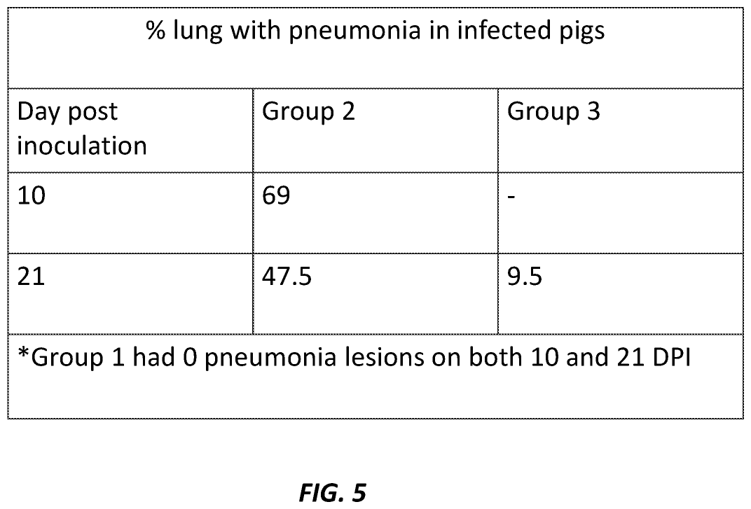

FIG. 5 shows that the percentage of lung samples with pneumonia in infected pigs was also less for the Group 3 pigs than the Group 2 pigs. At 10 DPI, none of the Group 3 pigs had pneumonia, while 69% of the Group 2 pigs did. By 21 DPI, the percentage of pneumonia-positive lungs in Group 3 pigs rose, but only to 9.5%, whereas the percentage of pneumonia-positive lungs in Group 2 pigs remained much higher, at 47.5%. Consumption of the add-pack thus delayed the onset of pneumonia in addition to reducing its incidence and severity. The delay may be critical for animals to build their immune system to the extent necessary to mitigate the condition. Accordingly, consumption of the add-pack reduced the incidence and severity of pneumonia in infected pigs relative to infected pigs not fed the add-pack.

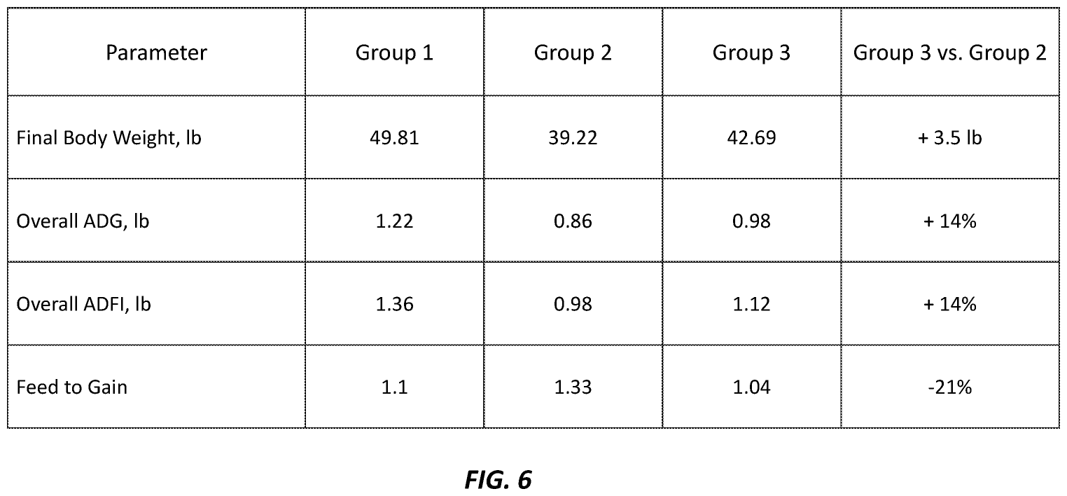

As shown in FIG. 6, consumption of the add-pack reduced the negative impact of PRRS and S. suis on growth performance, as evidenced by increased final body weight (+3.5 lbs.), increased average daily gain (+14%), increased average daily feed intake (+14%) and decreased feed-to-gain ratio (−21%) in the Group 3 animals relative to the Group 2 animals. Consumption of the add-pack thus increased final body weight, increased average daily gain, increased average daily feed intake, and reduced feed-to-gain ratios.

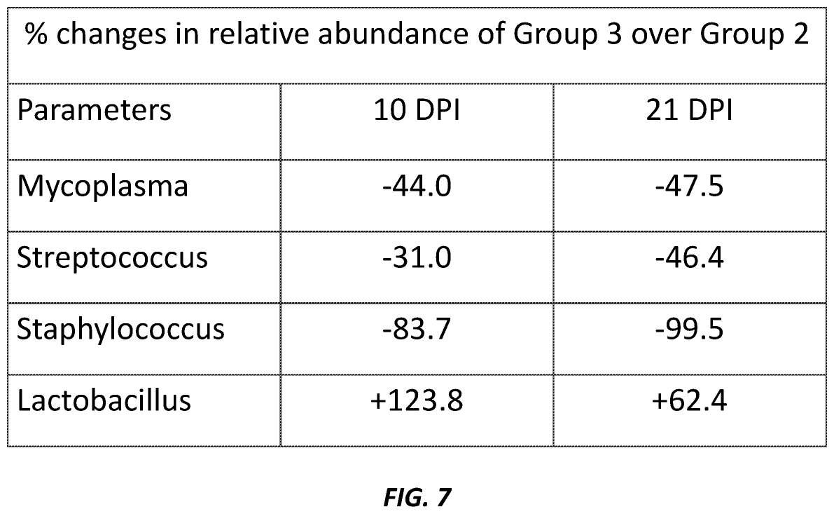

As shown in FIG. 7, the add-pack reduced the percentage of pathogenic bacteria and increased the percentage of beneficial bacteria in the test pigs at 10 DPI and 21 DPI. In particular, nasal swab samples obtained from Group 3 pigs comprised 44% lower and 47.5% lower abundance of Mycoplasma spp. relative to the Group 2 pigs at 10 and 21 DPI, respectively. Nasal swab samples obtained from the Group 3 pigs also included 31% lower and 46.4% lower abundance of Streptococcus than the Group 2 pigs at 10 and 21 DPI, respectively. Nasal swab samples obtained from the Group 3 pigs also included 83.7% lower and 99.5 lower abundance of Staphylococcus than the Group 2 pigs at 10 and 21 DPI, respectively. Still further, nasal swab samples obtained from the Group 3 pigs also included 123.8% greater and 62.4% greater abundance of beneficial Lactobacillus than the Group 2 pigs at 10 and 21 DPI, respectively. Accordingly, nasal swab samples obtained from animals fed the add-pack included decreased abundance of harmful Streptococcus, Staphylococcus, and Mycoplasma spp., and increased abundance of beneficial Lactobacillus. The add-pack thus promoted proliferation of beneficial bacteria, while suppressing proliferation of harmful bacteria in animals challenged with both PRRS and S. suis.

Trial 2

The objective of Trial 2 was to determine the impact of the feed product disclosed herein on wean-to-finish pigs obtained from a PRRSV-positive sow herd.

Materials and Methods. A total of 660 newly weaned pigs (DNA L600×241 genetics, average body weight of 16.0±0.32 lbs.) were divided into 33 pens (20 pigs per pen), with each pen consisting of an equal number of gilts and barrows. Each pen within a block (location within the barn) was randomly assigned to one of three dietary treatments consisting of either a non-supplemented basal diet (the negative control—“NC”), a basal diet admixed with a disclosed feed product comprising a combination of MCFAs, DFMs, and flavonoids (“Feed Product 1”), or a basal diet supplemented with a similar test feed product (“Feed Product 2”). The inclusion rates (lbs./ton of basal feed) of feed product 1 and feed product 2, by body weight and phase, are shown below in Table 1.

TABLE 2

| | NC | Feed Product 1 | Feed Product 2 |
|---|---|---|---|
| 15 to 25 lbs. BW (Phase 1-2) | 0 | 8 | 10 |
| 25 to 150 lbs. BW (Phase 3-6) | 0 | 4 | 5 |
| 150 lbs. to market weight (Phase 7-8) | 0 | 2 | 2.5 |

The trial comprised an eight-phase feeding program implemented from weaning until the pigs reached market weight. Each pig was fed about 2.5 lbs. of feed during phase 1, about 13.3 lbs. of feed during phase 2, about 40 lbs. of feed during phase 3, about 50 lbs. of feed each day during phase 4, about 110 lbs. of feed during phase 5, and about 120 lbs. of feed during phases 6 and 7. Phase 8 diets were fed until the end of the trial. All diets were manufactured as meal, except for the first two phases, which were made as pellets. The pigs had ad libitum access to feed and water throughout the trial. Individual pig weights were taken at the start and end of the trial. Pen weights and feed consumption were recorded at the end of each phase. Weights of dead pigs were also recorded.

In addition to obtaining a variety of growth and livability metrics (e.g., ADG, ADFI, feed-to-gain, and mortality), serum samples were collected from one sentinel pig per pen at 0, 21, 42, and 63 days post-weaning to determine the number of viremic pigs at each time point. Of the 33 pigs sampled at day 0, only those that survived until day 63 were accounted for at each time point. Real-time PCR was performed using the serum samples to determine the amount of virus in each sample, as indicated by the measured cycle threshold (CT) value. The percentage of PRRSV viremic pigs was calculated as the number of pigs with a CT value of less than 37 out of the total surviving pigs on day 63 for each feed treatment. Analyses of variance for growth and mortality were performed using Mixed Procedure in SAS (SAS 9.4, SAS Institute Inc., Cary, NC, USA) and a priori contrasts were used to detect treatment differences at P<0.05. Association between dietary treatments and the percentage of viremic pigs was evaluated for significance using Fisher's exact test at P<0.05.

Results. Providing feed product 1 and feed product 2 improved the overall growth performance of pigs. Pigs fed feed product 1 and feed product 2 had a final body weight advantage of +26 and +36 lbs., respectively, over the NC pigs, as shown in Table 3 (P<0.01). The average daily gain was also greater for pigs fed feed product 1 and feed product 2 (+13 and +18%, respectively; P<0.01) compared to the NC pigs. This positive response was driven at least in part by improvements in both feed intake and efficiency. Average daily feed intake was higher for the pigs fed feed product 1 and feed product 2 compared to the NC pigs (+7 and +12% for feed product 1 and feed product 2, respectively; P<0.01). Similarly, feed-to-gain ratios of pigs fed the test feeds were also lower compared to NC (−5.5 and −5.2 for feed product 1 and feed product 2, respectively (P<0.05).

TABLE 3

| | | Feed | Feed | | |
|---|---|---|---|---|---|
| | NC | Product 1 | Product 2 | SEM | P value |
| Initial BW | 16.1 | 15.8 | 16.0 | 0.32 | |
| D22BW | 25.1 | 25.6 | 25.6 | 0.59 | ns |
| D64BW | 80.7 | 80.4 | 83.4 | 2.06 | <0.05 |
| D106BW | 152.5 | 164.1** | 164.6** | 3.58 | <0.01 |
| Final BW | 219.3 | 245.0** | 255.2** | 5.13 | <0.01 |
| ADG, day 0- | 1.287 | 1.462** | 1.522** | 0.03 | <0.01 |
| market | | | | | |
| ADFI, day 0- | 3.478 | 3.735** | 3.906** | 0.15 | <0.01 |
| market | | | | | |
| Feed-to-gain, | 2.705 | 2.555** | 2.556 | 0.08 | <0.05 |
| day 0-market | | | | | |
| Morality & | 23.6 | 21.8 | 20.4 | 2.5 | ns |
| Removal %, | | | | | |
| day 0 to | | | | | |
| market | | | | | |

Accordingly, dietary supplementation with either feed product 1 or feed product 2 improved growth performance and livability of wean-to-finish pigs naturally infected with PRRSV, indicating that both feed products and associated feeding methods may boost immune responses to PRRSV and facilitate faster recovery from infection.

As used herein, the term “about” modifying, for example, the quantity of a component in a composition, concentration, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or components used to carry out the methods, and like proximate considerations. The term “about” also encompasses amounts that differ due to aging of a formulation with a particular initial concentration or mixture, and amounts that differ due to mixing or processing a formulation with a particular initial concentration or mixture. Where modified by the term “about” the claims appended hereto include equivalents to these quantities.

Similarly, it should be appreciated that in the foregoing description of example embodiments, various features are sometimes grouped together in a single embodiment for the purpose of streamlining the disclosure and aiding in the understanding of one or more of the various aspects. These methods of disclosure, however, are not to be interpreted as reflecting an intention that the claims require more features than are expressly recited in each claim. Rather, as the following claims reflect, inventive aspects lie in less than all features of a single foregoing disclosed embodiment, and each embodiment described herein may contain more than one inventive feature.

Although the present disclosure provides references to preferred embodiments, persons skilled in the art will recognize that changes may be made in form and detail without departing from the spirit and scope of the invention.

What is claimed is:

1. A method of mitigating pathogenic infection in an animal, the method comprising:
admixing a mitigant composition with a base feed to form a mitigant feed mixture, wherein the mitigant composition comprises a medium chain fatty acid composition and a direct-fed microbial composition,
wherein the direct-fed microbial composition comprises two or more strains of Bacillus selected from strains of Bacillus pumilus, Bacillus subtilis, and Bacillus amyloliquefaciens,
wherein the medium chain fatty acid composition comprises one or more of caprylic acid, capric acid, or lauric acid; and
providing the mitigant feed mixture to the animal in an amount effective to mitigate the pathogenic infection.

2. The method of claim 1, wherein the mitigant feed mixture is provided on an ad libitum basis.

3. The method of claim 1, wherein the mitigant composition further comprises one or more flavonoids comprising a citrus flavonoid.

4. The method of claim 3, wherein the animal is a nursery pig.

5. The method of claim 1, wherein the mitigant composition further comprises one or more organic acids.

6. The method of claim 5, wherein the one or more organic acids comprise benzoic acid.

7. The method of claim 1, wherein the pathogenic infection comprises a porcine reproductive respiratory syndrome virus infection.

8. The method of claim 1, wherein the pathogenic infection comprises a Streptococcus suis infection.

9. The method of claim 1, wherein the base feed comprises a complete swine feed, blood meal, porcine meat and bone meal, spray-dried animal plasma, feather meal, avian blood meal, poultry by-product meal, vitamin D, lysine hydrochloride, choline chloride, and/or soybean meal.

10. The method of claim 1, wherein the two or more strains of Bacillus are included in approximately equal amounts.

11. The method of claim 1, wherein the direct-fed microbial composition constitutes about 0.1 wt % to about 5.0 wt % of the mitigant composition.

12. The method of claim 1, wherein the medium chain fatty acid composition constitutes about 20 wt % to about 60 wt % of the mitigant composition.

13. The method of claim 1, wherein the animal ingests about 0.1 g to about 5.0 g of the mitigant composition per day.

14. The method of claim 1, wherein the inclusion rate of the mitigant composition in the base feed is about 1 lb./ton to about 20 lbs./ton.

15. The method of claim 1, wherein providing the mitigant feed mixture to the animal in an amount effective to mitigate the pathogenic infection comprises providing the mitigant feed mixture to the animal in an amount effective to reduce one or more symptoms of PRRS in the form of respiratory distress, weight loss, decreased feed intake, or increased feed-to-gain ratio.

16. The method of claim 1, wherein providing the mitigant feed mixture to the animal in an amount effective to mitigate the pathogenic infection comprises providing the mitigant feed mixture to the animal in an amount effective to reduce PRRS viral load in serum and lung tissue of the animal.

17. A feed product formulated for swine, the feed product comprising:
a complete base feed comprising one or more of: ground corn, sugar, dehulled soymeal, soy isolate, hydrolyzed soy protein, dried distillers grains with solubles, poultry byproduct meal, vitamins, or minerals;
at least one direct-fed microbial comprising two or more strains of Bacillus selected from strains of Bacillus pumilus, Bacillus subtilis, and/or Bacillus amyloliquefaciens; and
at least one medium chain fatty acid comprising one or more of caprylic acid, capric acid, or lauric acid.

18. The feed product of claim 17, further comprising one or more flavonoids comprising a citrus flavonoid.

19. The feed product of claim 17, further comprising one or more organic acids comprising benzoic acid.

20. The feed product of claim 17, further comprising one or more anti-caking agents, flavorants, desiccants, emulsifiers, preservatives, stabilizers, salts, or combinations thereof.
